(19) United States
(12) Patent Application Publication
(10) Pub. No.: US 2013/0023980 A1
(43) Pub. Date: Jan. 24, 2013

(54) Oval Aortic Valve

(71) Applicant: William Joseph Drasler, Minnetonka, MN (US)

(72) Inventor: William Joseph Drasler, Minnetonka, MN (US)

(21) Appl. No.: 13/549,726

(22) Filed: Jul. 16, 2012

Related U.S. Application Data
(60) Provisional application No. 61/572,849, filed on Jul. 22, 2011.

(51) Int. Cl. A61F 2/06

(52) U.S. Cl. 623/1.26

(57) ABSTRACT

An oval valve for use in transcutaneous aortic (TAVI) or mitral valve implantation or for direct access valve implantation. The oval leaflet frame or stent provides a better seal with the oval native annulus to reduce perivalvular leaks. The valve leaflets are a bileaflet configuration to provide improved leaflet coaptation independent of the amount of ovality of the native valve annulus. The bileaflet configuration is less dependent upon the diameter and perimeter of the native valve annulus and provides leaflet coaptation without intravalvular leakage.

### CROSS REFERENCE TO RELATED APPLICATIONS

This patent application makes reference to and thereby incorporates all information found in issued U.S. Pat. Nos. 6,245,101 and 6,451,051 which describe aspects of stents and attachment means having hinges and struts. This patent application makes reference to and incorporates all information found in the provisional patent application No. 61/572,849 entitled Oval Aortic Valve, filed 22 Jul. 2011 by William J. Drasler.

### FIELD OF THE INVENTION

This invention relates to transcatheter aortic valve implantation (TAVI) devices or direct surgical access devices that push the native aortic valve leaflets to the side and cover the native aortic valve leaflets. The TAVI devices are comprised of a stent onto which is mounted a flexible leaflet valve. The TAVI device is generally delivered via access from the femoral artery, or through apical access, through direct access into the aorta, or via other large vessels that are suitable for large catheter access. The present invention can similarly be used for percutaneous, transcutaneous, or direct access replacement of a stenotic or refluxing mitral valve.

### BACKGROUND OF THE INVENTION

Surgical implantation of aortic valves is the method of choice for patients having aortic valve stenosis and who are candidates for surgical valve implantation. For those patients that are not well suited to undergo valve surgery, the aortic valve can be implanted via a vastly less invasive procedure either via femoral access, apical access, or other large vessel access. Other valves such as the mitral valve can similarly be implanted via less invasive trascutaneous methods. The TAVI device is delivered through a catheter in a small diameter configuration and the stented TAVI device is expanded in place to push the stenotic aortic valve leaflets aside. The stent portion of the TAVI device can be either a balloon expandable or a self-expanding stent. Attached to the stent of the standard TAVI device are three tissue leaflets that generally resemble the structure of a healthy semi-lunar trileaflet aortic valve found in most healthy humans. The balloon expandable stent is expanded out via a round balloon to form a generally round cross-sectional shape for the stent or frame to push the native leaflets to the side and to hold the stent firmly in place against the old stenotic valve leaflets and the valve annulus and prevent embolization of the valve. The round shape allows the three leaflets of the valve to coapt with each other and generally prevent reflux of blood through the leaflets. The self-expanding stent also expands out to a round shape to hold the newly implanted tissue leaflets in a round configuration necessary to obtain coaptation of the leaflets with each other and provide a high level of force outwards of the stent against the old stenotic leaflets and the valve annulus.

The shape of the aortic annulus for a patient undergoing the TAVI procedure is oval. The long axis of the oval tends to run in a direction in line with the direction of the anterior mitral valve leaflet. Thus when a round stented valve is placed into this oval configuration, two issues arise that reduce the performance of the TAVI device. First, the stent of the TAVI device can begin to take on a slight oval shape and thereby cause reflux in the typical trileaflet valve due to a lack of tight coaptation of the leaflets with each other. Second, the seal of the round stented valve with the oval-shaped annulus leaves a gap at each end of the oval at the end of the long axis; often calcium deposits are located here to further increase the amount of blood reflux or regurgitation at this site. Reflux of blood through inadequately coapted leaflets or perivalvular leaks around the stent due to the oval shaped annulus can lead to aortic valve regurgitation, left ventricular heart failure, and possibly death. An improvement is needed to ensure that the TAVI devices are able to better fit within the oval space provided by the stenotic aortic valve and the oval annulus.

### SUMMARY

The present invention is a TAVI device having a stent or frame that has an oval shape. To allow the oval-shaped TAVI device to function regardless of whether the annulus is highly ovalized or whether it is almost round, the trileaflet configuration used in existing devices has been replaced by a bileaflet design. The TAVI device can be used for either aortic valve or mitral valve implantation. The expandable valve of the present invention is well suited for implant as a mitral valve replacement. In a manner similar to that described for the aortic valve, the mitral valve is placed over the native stenotic or incompetent native mitral valve leaflets and attached to the mitral valve ring. The bileaflet design of the present invention is better suited to allow contraction of the left ventricle than a trileaflet design without compromising coaptation of the bileaflet configuration of the present invention. This patent application will describe and focus primarily on the aortic valve application although the design applies equally to both mitral valve and aortic valve applications.

The expandable aortic valve of the present invention is delivered to the patient via a percutaneous catheter placed into the femoral artery, the axillary artery, subclavian artery, aorta, other large vessel, or via a thoracotomy into the patient's chest and delivery through the apex of the heart. Once the expandable aortic valve is placed adjacent to the stenotic native valve leaflets, it is expanded to place the aortic valve of the present invention on top of the native valve leaflets pushing the native leaflets to the side.

One element of the present invention is a metal frame or stent that is expanded to hold the native valve leaflets outwards and prevents embolization of the TAVI device. The metal frame can be a self-expanding material such as Nitinol or it can be balloon expandable such as stainless steel, Cobalt Chrome alloy, or other metal or polymeric material used in stent design. The metal frame is designed such that it achieves an oval shape when it is expanded out from a small diameter configuration to a large diameter configuration. For the balloon expandable frame, the frame design controls expansion along the long axis of the stent such that it forms an oval shape upon expansion via a balloon. The self-expanding frame is thermally processed in an oval shape such that it retains its oval shape upon reexpansion to a large diameter configuration.

Attached to the metal frame are two flexible leaflets such as tissue formed leaflets, synthetic materials, composite leaflet materials, or other deformable or flexible leaflet; thus the valve of the present invention is a bileaflet aortic valve. The bileaflet valve will allow efficient coaptation of the free edges of the leaflets without detrimental effect due to the formation of an oval shape. The standard trileaflet valve design is negatively impacted when it is unduly forced into an oval shape resulting in intravalvular leakage of blood.

The bileaflet design also allows the diameter (or perimeter) of the expandable valve of the present invention to be increased or decreased significantly and still maintain efficient coaptation of the free edges and adjacent marginal leaflet surfaces of the leaflets. This will allow the physician to further expand the metal frame or stent within the annulus to make a fluid tight seal around the perimeter of the valve, thereby obviating the propensity for forming perivalvular leaks regardless of whether a larger diameter or perimeter valve or a smaller diameter or perimeter valve is warranted. The bileaflet configuration of the present invention will also improve leaflet coaptation over a wide range of annular ovality that has a major axis 5-35 percent greater than its minor axis, or more. The ratio of the major axis to the minor axis for the frame of the present invention ranges from 1.05-1.35 and can preferably, for example, have a ratio of 1.10-1.25. The bileaflet configuration will reduce the amount of intravalvular leaks that occur between the leaflets. Such intravalvular leaks can also occur when a valve of too large of a perimeter is placed into a native annular perimeter that is of a lower diameter or perimeter. The bileaflet configuration of the present invention can provide a greater leaflet coaptation with less intravalvular leakage over a greater range of valve perimeters than a trileaflet valve configuration such as currently being used in the clinic.

The oval frame with the bileaflet valve design also allows an improved fit between the oval metal frame of the present invention and the oval shape of the aortic annulus thereby further reducing perivalvular leaks. The standard round stents used in current TAVI devices allow leakage of blood around the standard circular stent at each end of the long axis of the oval shaped annulus.

The oval frame and the bileaflet design of the present invention will provide a more uniform application of outward force from the stent frame against the aortic annulus to ensure a good seal and also to prevent embolization of the stented valve. This uniform application of force will allow the local force applied at any specific location along the perimeter of the stent to be less than if the stent only interfaced with the annulus as specific or focal spots along its perimeter. This lowering of the outward force requirement to maintain a good seal with the annulus and prevent embolization will have a benefit at reducing the incidence of heart block due to excessive force application onto the membranous septum or the left bundle branch.

The oval frame can be positioned such that the valve commissures are not placed at a location that could interfere with the left or right coronary arteries found in the aortic sinus. In one embodiment the commissures are located aligned with the long axis of the oval shape of the annulus and each leaflet is of similar size to each other. Alternately, in another embodiment the commissures are aligned with the short axis of the oval shape. In yet another embodiment one of the leaflets of the bileaflet aortic valve of the present invention can be larger than the other in a manner similar to that found in the bileaflet native mitral valve leaflets or some native bileaflet aortic valve leaflets. The larger size leaflet can be longer in the long axis direction or in the short axis direction of the oval. In yet another embodiment, the commissures of the bileaflet valve of the present invention are aligned with the short axis of the aortic annulus and are of similar size. Alternately, each of the two leaflets can be aligned with the short axis be of a different size from each other.

In still another embodiment of the present invention, the frame is made with a wall structure that includes hinges and struts with a special dimensioning. The hinge width is narrower than the strut width such that the hinge flexes during expansion of the frame and the strut does not. The strut depth is smaller than the hinge depth such that the strut flexes elastically during a shape change to an oval shape in its fully expanded configuration. The hinge does not flex during such an oval deformation. The hinge length is very short in comparison to the strut length such that during a balloon expansion the hinge formed from the balloon expandable frame material will deform plastically while the strut will remain elastic during a crush deformation due to the thin depth of the strut. The hinge length extends from one transition region to another and undergoes substantially all of the deformation that occurs during the expansion deformation of the stent during deployment. For a self-expanding stent the stent can retain a large outward force as controlled by the hinge depth and width while the strut allows for very soft flexure due to its thin depth in order to form an oval shape.

### BRIEF DESCRIPTION OF THE DRAWINGS

FIG. 1A is a plan view of the native valves of the heart showing the oval shape for the aortic annulus and having a bileaflet valve aortic valve of the present invention implanted within.

FIG. 1B is a plan view of an example of a round aortic annulus with a native trileaflet valve.

FIG. 2 is a plan view of a heart, aortic sinus, and aorta having a transcutaneous aortic valve implanted across the aortic valve an holding the native aortic valve leaflets outward against the aortic sinus.

FIG. 3A is a plan view of a replacement bileaflet valve leaflet showing the marginal surface that would coapt against another leaflet.

FIG. 3B is a perspective view of a replacement bileaflet showing a nonplanar surface with a pocket.

FIG. 4A is a perspective view of frame containing two leaflets of a bileaflet valve attached to the frame and showing the long and short axis of the valve and the frame.

FIG. 4B is a top view of the bileaflet valve of FIG. 4A and showing the oval aortic annulus.

FIG. 5A is a plan view of a bileaflet aortic valve implant having leaflets with differing minor axes and differing surface areas from each other and having the long axis of the leaflet aligned with the long axis of the frame.

FIG. 5B is a plan view of another embodiment of a bileaflet aortic valve having a larger anterior leaflet surface area.

FIG. 6A is a plan view of a bileaflet valve having the long axis of the leaflet aligned with the short axis of the valve frame and similarly sized leaflets.

FIG. 6B is a plan view of a bileaflet valve having the long axis of the leaflet aligned with the short axis of the valve frame and having leaflets of differing surface areas between leaflets.

FIG. 7A is a plan view of a frame having an oval shape but having a trileaflet valve wherein two of the leaflets are smaller than the third leaflet.

FIG. 7B is a plan view of a round frame having a bileaflet valve attached.

FIG. 7C is a perspective view of a frame with a bileaflet valve attached to it and having a skirt located on the outside surface of the frame.

FIG. 8A is a plan view of a frame or stent for a transcutaneous aortic valve having a wall structure with a zig zag structure with expansion limiters in a nonexpanded configuration.

FIG. 8B is a plan view of a frame or stent for a transcutaneous aortic valve having a wall structure with a zig zag structure with expansion limiters in an expanded configuration.

FIG. 9A is a perspective view of a frame or stent for a transcutaneous aortic valve showing a section of the frame and its wall structure.

FIG. 9B is a plan view of a wall structure for a frame having a zig zag structure with a specialized hinge and strut wall structure.

FIGS. 9C-E are plan views of the wall structure for a frame having the specialized hinge and strut structure.

### DETAILED DESCRIPTION OF THE INVENTION

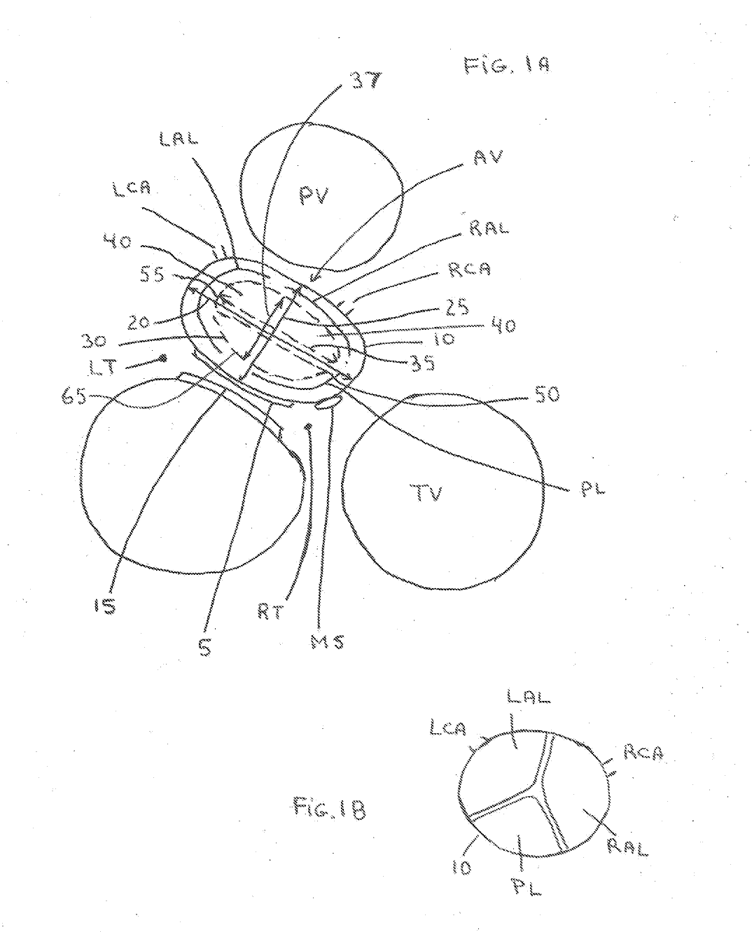

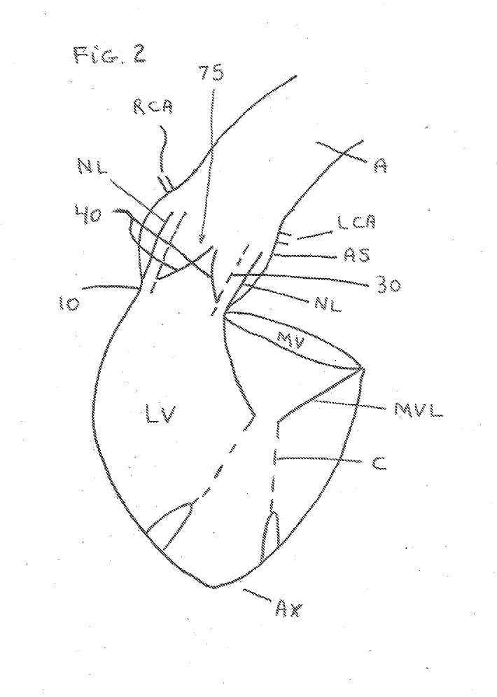

Shown in FIGS. 1A, 1B and 2 are views of a heart and the valves made through an approximate plane which very nearly passes through the fibrous rings that form the bases of attachment for the four major valves found in the heart, the mitral valve (MV), tricuspid valve (TV), aortic valve (AV) and the pulmonary valve (PV). The posterior aspect (5) of AV annulus (10) comes into an elongated contact with the anterior aspect (15) of the mitral valve (MV) ring; the aortic annulus (10) is elongated to form an oval with the annulus long axis (20) along the direction of this elongated contact extending from the right trigone (RT) to the left trigone (LT); the annulus short axis (25) is perpendicular to the annulus long axis (20).

The three native aortic valve leaflets (40) are generally positioned such that the native right anterior leaflet (RAL) is located on the anterior right aspect of the aortic annulus (10) and is closely associated with the right coronary artery (RCA). The native left anterior leaflet (LAL) is located on the anterior left aspect of the aortic annulus (10) and is associated with the left coronary artery (LCA). A third native posterior leaflet (PL) does not have a coronary artery associated with it. A membranous septum (MS) is located between the right atrium which is just located above the tricuspid valve (TV) and the left ventricle (LV) located below the mitral valve (MV) and aortic valve (AV).

The native mitral valve (MV) structure is normally comprised of a native bileaflet valve in order to accommodate the contraction of the left ventricle (LV) during systole and to adjust for the large changes in diameter that occur for the left ventricle that then occur during diastole. These native bileaflet mitral valve leaflets (MVL) have a structure, however, that requires it to have cordae (C) attached to its free edge (65) in order to prevent it from prolapsing and resulting in blood reflux. The present invention is a bileaflet valve (75) that does not require such cordae but instead relies on a cup shaped leaflet structure and commissures that will be described further during discussion of the aortic valve application.

The present invention is an oval-shape expandable valve that is delivered to a location adjacent to the stenotic native aortic valve leaflets (RAL, LAL, and PL) in a small diameter round configuration and expanded to a larger diameter oval configuration as shown in FIGS. 1A, 1B, and 2. The TAVI catheter can be provided access to the vasculature via the femoral artery and delivered retrogradely through the aorta until the frame (30) is located adjacent to the aortic annulus (10) and native leaflets found in the aortic sinus (AS). The TAVI catheter can also be delivered through a thoracotomy and into an access hole made near the apex (AX) of the left ventricle (LV) and into position adjacent to the annulus (10) and the stenotic aortic valve native leaflets (NL). Other surgical and interventional methods can be used to deliver the present invention to the aorta (A) via other large vessels of the body. The frame (30) of the present expandable stented oval aortic valve can be expanded via standard balloon dilation as currently used in standard TAVI procedures. The materials of such a frame (30) can be stainless steel, cobalt chrome alloy, or other metals commonly used for stent devices. Alternately the frame (30) can be made of an elastic metal such as Nitinol and can be released from an external sheath to expand out to its natural equilibrium diameter and shape. The diameter of the frame (30) ranges from approximately 15-38 mm in diameter.

The oval frame (30) of the present invention is positioned such that the frame long axis (35) aligns with the annulus long axis (20). Two implant or replacement leaflets (40) of one embodiment are attached to the internal surface (45) of the oval frame (30) as shown in the embodiment of FIGS. 1 and 2 with the right commissures (50) and left commissures (55) of the leaflets (40) aligned with the frame long axis (35). The ovality of the aortic annulus (10) can be expressed as a ratio of the major or long axis (20) to the minor or short axis (25) or percentage that the major or long axis (20) exceeds the diameter of the minor or short axis (25). Often the annulus (10) ovality is approximately 5-25 percent larger in the annulus major axis (20) than the minor axis (25); the frame of the present invention can similarly have a frame major or long axis (35) that is 5-25 percent larger in the frame minor or small axis (45). The ovality of the aortic annulus (10) can be even greater that 25 percent is some patients and can reach a 35% larger annulus major axis (20) than minor axis (25) (i.e., a major to minor axis ratio ranging from 1.05 to 1.35) or greater; the valve frame (30) can similarly be of greater ovality, with a frame major or long axis (35) that is 5-35% larger than the frame minor or short axis (37). A typical level for the ovality of the annulus and frame is 10-20% larger in the long axis than the short axis or having a ratio of 1.10-1.20 although this ratio can go higher than even 1.35. The larger the ovality of the annulus (10) and hence the ovality of the frame (30) of the present implanted valve, the greater the benefit from the present invention having an oval frame (30) to provide a better fit of the frame into the annulus.

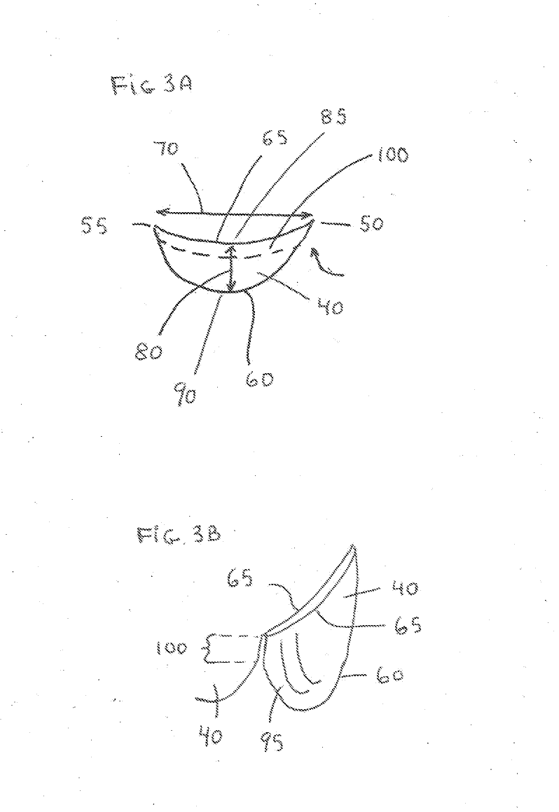

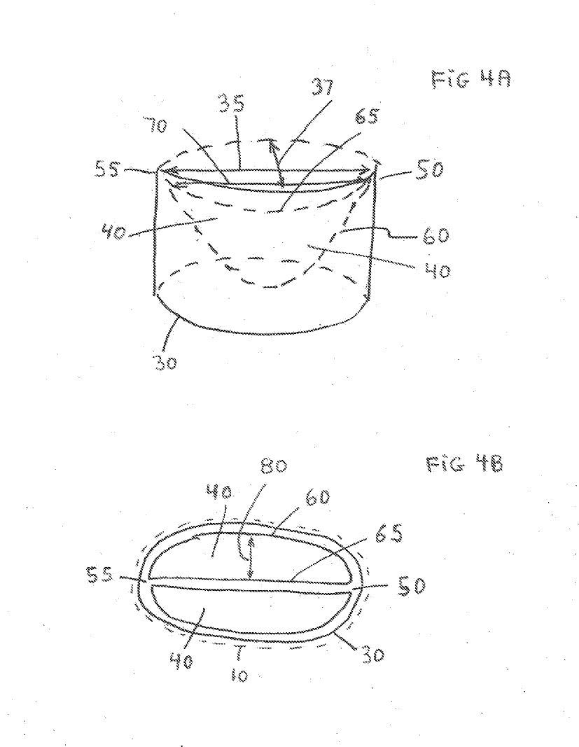

Each leaflet of the present bileaflet aortic valve is formed with a crescent shape as shown in FIGS. 3A and 3B. The leaflet has an attached edge (60) that is attached along its entire length to the frame (30) to ensure that blood leakage or reflux is obviated. A free edge (65) extends from a left commissure (55) or one commissural end of the leaflet to a right commissure (50) at the other commissural end of the leaflet. The leaflet longitudinal axis extends from the left commissure (55) to the right commissure (50) or from one commissural end to the other commissural end. The bileaflet valve (75) of the present invention can extend or reduce in length more easily from commissure to commissure while still maintaining a good leaflet coaptation (i.e., the length of the long axis can change, for example, and still maintain good leaflet coaptation). The leaflet short axis (80) extends from a concave point (85) on the free edge (65) to the convex point (90) on the attached edge (60). The leaflet does not form a planar shape; the leaflet is formed with a small pocket or pouch (95) in the surface of the leaflet. This pocket or pouch (95) allows the free edge (65) plus additional marginal surface (100) to coapt with its neighboring leaflet to make an effective seal. This seal of the pockets allows good coaptation of the bileaflet design even though there may be change in the diameter along the direction of the pocket (for example in the direction of the short axis). The leaflet long axis and leaflet short axis can have a ratio that allows it to fit into the frame (30) with a frame long axis that is 5-25 percent longer than the frame short axis. The ratio of the leaflet long axis to twice the leaflet short axis (80), as shown in FIG. 4B in the direction of the plane of the aortic annulus (10) has a ratio that is 5-25% longer in the leaflet long axis (70) than twice the leaflet short axis (i.e., a ratio of 1.05-1.25 to 1.0). Often the annulus and hence the frame will have a long axis that is approximately 10-20% larger than the short axis.

The leaflet can be formed from pericardial tissue such as bovine, equine, or porcine pericardium or it can be formed from other tissues taken from allogeneic, heterogeneic, xenogeneic sources. Alternately, the implant or replacement leaflets (40) can be formed from synthetic materials or from a composite structure that includes both tissue and synthetic materials including PTFE, ePTFE, Dacron, polyurethane, Nylon, Nitinol, stainless steel, and other flexible materials suitable for implants.

In one embodiment the implant or replacement leaflets (40) are attached to the frame (30) as shown in FIGS. 4A and 4B; the frame long axis (35) is aligned with the leaflet long axis (70). The attached edges (60) of the leaflets (40) are attached to the frame (30) along the entire length of the attached edge (60). The left commissure (55) and right commissure (50) of one leaflet are adjacent the left commissure (55) and right commissure (50) of the second leaflet at each end of the frame long axis (35) and the leaflet long axis (70). The leaflet commissure is the junction of one leaflet free edge (65) with the free edge (65) of another leaflet. In this embodiment the right implant or right commissure (50) is located at the junction of the leaflet free edges (65) of each leaflet with the frame long axis (35) near the tricuspid valve (TV). The left commissure (55) is located on the opposite side of the frame (30). In this embodiment each of the implant or replacement leaflets (40) of this bileaflet valve (75) are the same size in the leaflet long axis (70) and in the leaflet short axis (80).

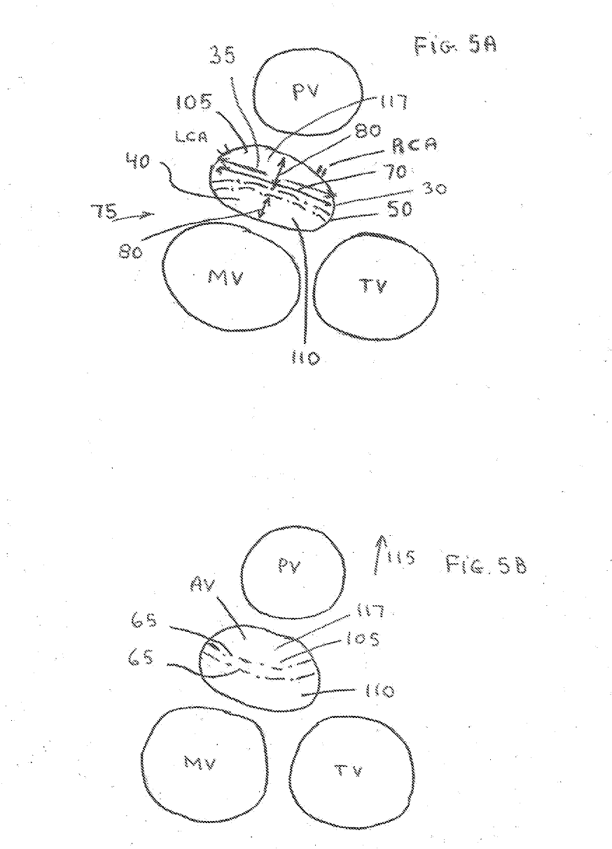

The implant leaflets (40) of the present invention are not required to have the same size both in their dimensions and in their surface area. FIG. 5A shows one embodiment where that anterior leaflet (105) is larger in the leaflet short axis (80) than the leaflet short axis (80) of the posterior leaflet (110). The anterior leaflet (105) of this embodiment has a larger leaflet surface area (117) than the posterior leaflet (110). The leaflet free edge (65) of this embodiment when viewed from the top as in FIG. 5A is not straight but is convex toward the anterior direction (115) of the aortic valve. The free edges (65) of the leaflets (40) could alternately be straight when viewed from the top. The commissures (50 and 55) of the bileaflet valve (75) leaflets (40) are also not required to be coincident with the frame longitudinal axis.

FIG. 5B shows an alternate embodiment for the shape of the leaflets (40) wherein the anterior leaflet (105) is smaller in leaflet surface area (117) than the posterior leaflet (110) and the free edge (65) is concave with respect to the anterior leaflet (105) of the aortic valve.

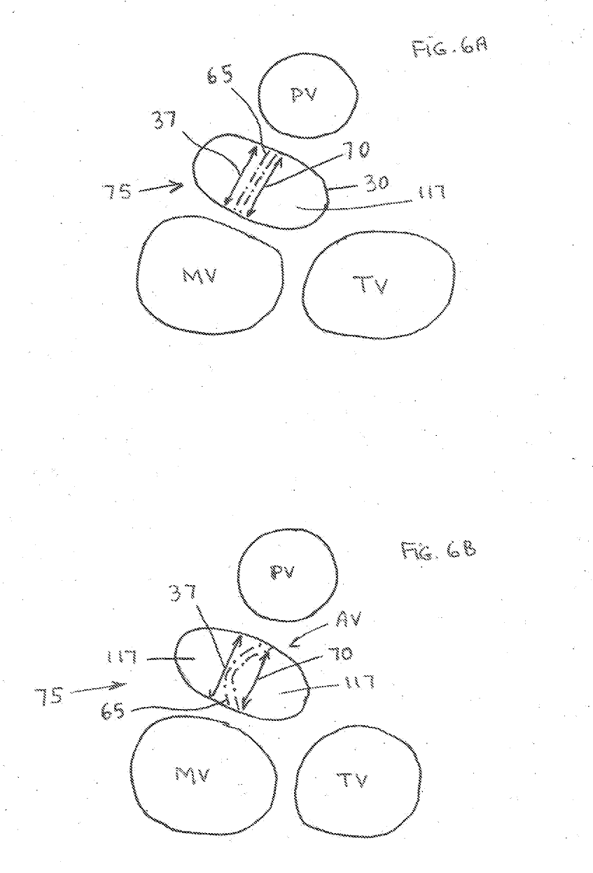

FIG. 6A shows yet another embodiment of the bileaflet valve (75) of the present invention having each of the leaflets (40) oriented such that their long axis (between commissures along the free edge) is aligned with the frame short axis (37). In this embodiment each of the leaflets (40) are of the same size in both the long and short axis and in leaflet surface area (117) for each leaflet.

FIG. 6B shows yet another embodiment of the present invention wherein each of the leaflets (40) of the bileaflet valve (75) are oriented with their long axis (along the free edge) aligned with the frame short axis (37) but one of the leaflets (40) has a larger leaflet surface area (117).

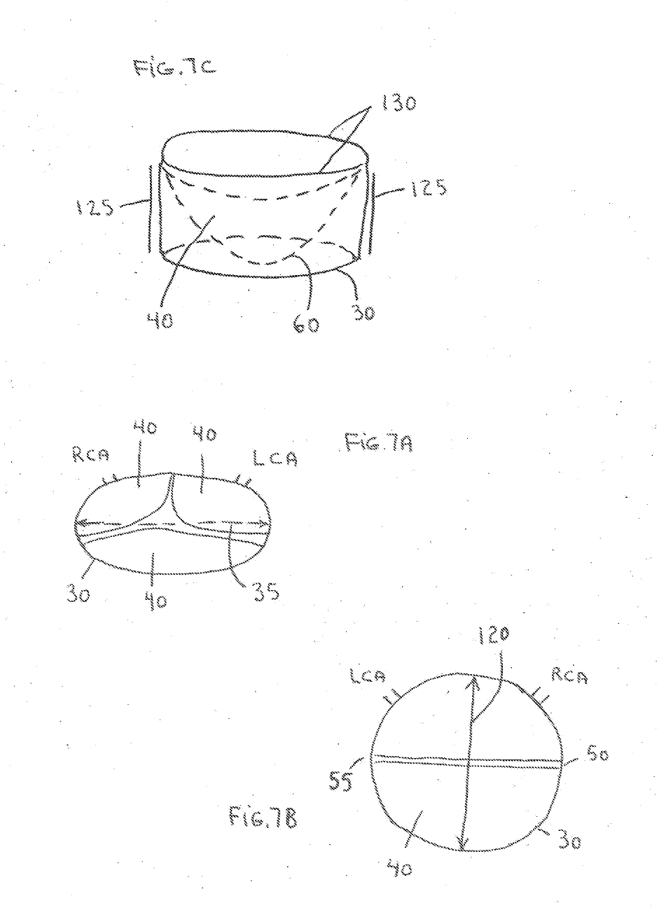

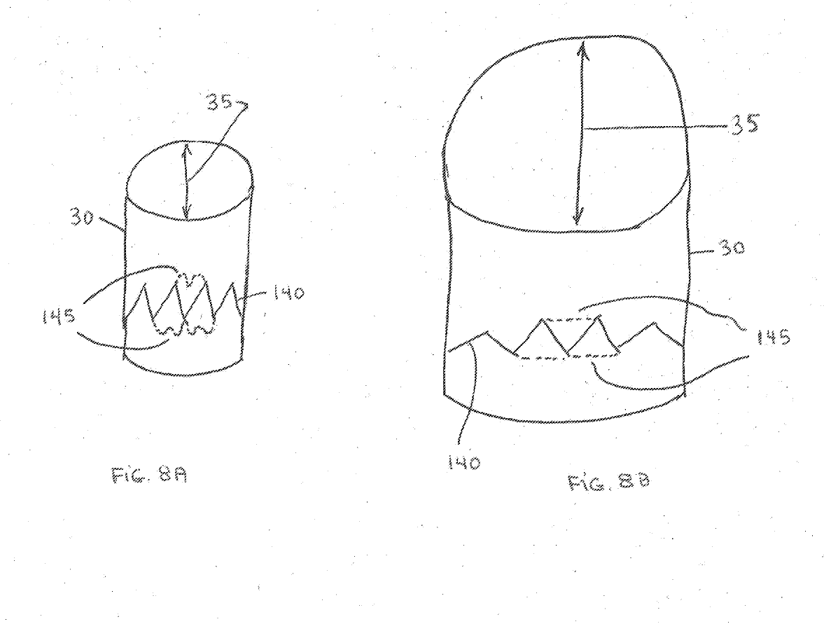

In yet another embodiment of the present invention, a trileaflet valve can be formed into an oval frame (30) as shown in FIG. 7A. The oval frame (30) will fit more efficiently within the oval-shaped annulus (10) without resulting in perivalvular reflux. The location of the three leaflets (40) along the frame long axis (35) and frame short axis (37) can be adjusted in size and length to provide adequate coaptation of the leaflets (40). As seen in the embodiment of FIG. 7A, for example, the leaflet adjacent to the LCA and the leaflet adjacent to the RCA are smaller than the third leaflet not adjacent to a coronary artery.

In a further embodiment, a round frame (30) can be used with a bileaflet valve (75) structure as shown in FIG. 7B. The diameter of this structure will be less dependent upon the frame diameter (120) and the annulus (10); the effective or average diameter of the annulus as determined by the diameter of a circle with the same perimeter is not always accurately measured before delivering the stented valve implant to the site of the native valve and annulus (10). This embodiment can be more fully expanded into an annulus (10) thereby forming a better perivalvular seal and reducing perivalvular leaks without affecting coaptation of the leaflets (40). Also, this valve embodiment can be expanded to a smaller diameter than its intended diameter or optimal frame diameter (120) (based on matching the frame perimeter (130) with the annulus perimeter) and still maintain good valve leaflet coaptation.

Any of the embodiments of the present invention can have a skirt (125) or fabric material attached to the frame (30) along the perimeter (130) of the frame (30) as shown in FIG. 7. The skirt (125) is intended to ensure that blood does not have a pathway for leakage from the space between the leaflet and the frame (30). Materials for the skirt (125) include expanded polytetrafluoroethylene, Dacron fabric, polyurethane fabric, or other thin but strong polymeric or tissue based material that can be attached to the outside or along the perimeter (130) of the frame (30) or to the attached edge (60) of the leaflet. Other reference numerals represent structure presented in previous figures.

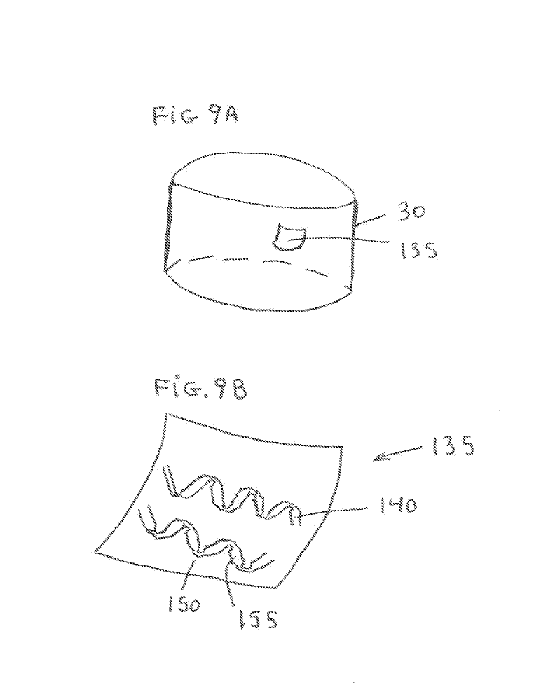

The balloon expandable oval frame (30) of the present invention can achieve an oval shape upon expansion to a large diameter configuration by providing a wall structure (135) such as shown in FIG. 8A in a nonexpanded configuration and FIG. 8B in an expanded configuration. In this wall structure (135) the frame (30) has a generally zig zag structure (140) that expands upon application of an outward force by an internally placed balloon. Expansion limiters (145) can be placed into the zig zag wall structure (135) of the frame (30), for example, in a region of the wall structure (135) that is on each end of the frame long axis (35). The expansion limiters (145) retain the stent in an elastic state thereby preferentially preserving the shape of its smaller nondeployed state. Upon expansion of this stent or frame (30) to a larger round shape, the expansion limiters will prevent that region containing the limiters from undergoing plastic deformation and will therefore remain elastic. All other regions not occupied by expansion limiters (145) will plastically deform during expansion and will retain their final shape. Upon deflation of the balloon, the frame (30) will have an oval shape with the expansion limiters (145) located at each end of the frame long axis (35). Alternately, any flexible balloon-expandable stent design can be used and inflated within the oval annulus (10) and will assume an oval shape post dilation.

The self-expanding frame (30) of the present invention can be formed into an oval via thermal processing steps know in the industry for retaining a shape such as an oval shape in an elastic metal such as Nitinol. The oval frame (30) can be delivered within an external sheath to the site of the stenotic native heart valve. Upon removal of the sheath, the frame (30) will expand outward to form a large oval shape and push the stenotic leaflets against the aortic sinus. The two leaflets (40) that are attached to the frame (30) will form a new bileaflet valve (75) that can assume the oval shape provided by the oval-shaped annulus (10). A post dilation step can be applied within the frame (30) using a balloon catheter to further dilate the frame (30) into contact with the aortic annulus (10) and the native valve leaflets.

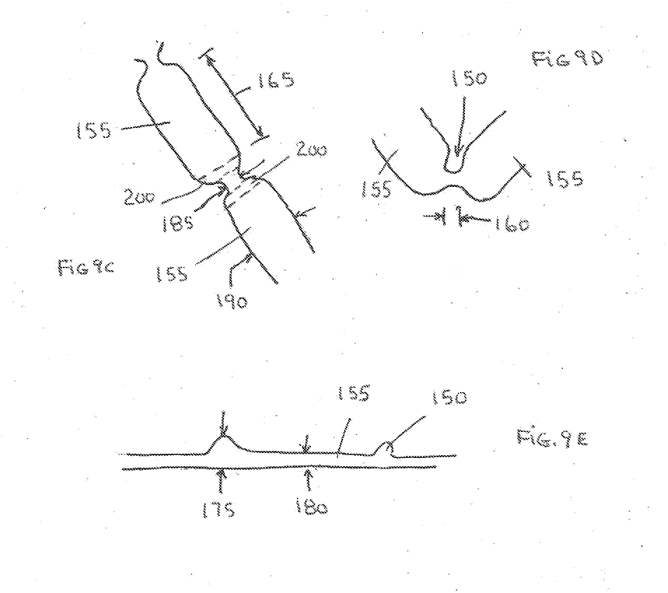

An alternate embodiment for forming a wall structure (135) for the frame (30) of the present invention is shown in FIGS. 9A-9E; reference is made to issued U.S. Pat. Nos. 6,245,101 and 6,451,051 for further description of the hinge (150) and strut (155) structure. The specialized hinge (150) and strut (155) wall structure (135) for the frame (30) seen in FIG. 9A can be a zig zag structure (140) such as that shown in FIG. 9B having specialized hinges (150) and specialized struts (155). The hinge (150) has a hinge length (160) that is less than ½ the length of the strut length (165) as shown in FIGS. 9C-E. The hinge (150) extends from one transition region (170) to another and undergoes substantially all of the deformation that occurs during expansion deformation as shown in FIG. 9D. This short hinge length (160) causes a balloon expandable frame (30) material to focus its deformation during expansion at the hinge (150) such that it deforms plastically. The long strut length (165) allows the strut (155) to bend over a long length when it bends to a curved shape caused by a crushing deformation. This long strut length (165) helps to maintain the strut (155) in an elastic state during such deformation. The hinge depth (175) is greater than the strut depth (180), and the strut depth (180) is maintained very thin as shown in FIG. 9E. This allows the strut (155) to bend elastically during a crush deformation even if the frame (30) is constructed out of a plastically deformable material such as stainless steel; the hinge (150) will not bend at all during such a crush deformation due to the relatively larger hinge depth (175). The hinge width (185) is very much smaller than the strut width (190) as shown in FIG. 9C such that during the expansion deformation, the hinge (150) will focus its deformation and will deform plastically while the strut (155) will not bend at all in the direction of the strut width (190) during such expansion deformation. The transition region (170) has a transition depth (195) and a transition width (200) that ranges from those of the hinge (150) to the strut (155). The transition regions (170) do not bend substantially during expansion deformation or during a crush-type of deformation of the wall structure (135).

The wall structure (135) with the specialized hinges (150) and struts (155) can be formed into a balloon expandable or a self-expanding frame (30) for the present invention. As a self-expanding frame (30) the frame can be designed to have hinges (150) of very large hinge depth (175) to provide a significant amount of expansion force to hold the frame (30) outward against the annulus (10). The struts (155) can be formed with very thin or small strut depth (180) to allow the frame (30) to deform easily to an oval shape with very little force. The present specialized hinge (150) and strut (155) structure can alternately produce a self-expanding frame (30) that will always remain round by providing a large strut depth (180) that deforms uniformly but with a large circumferential hoop strength or force.

As a balloon expandable frame (30) the specialized hinge (150) and strut (155) design can provide a hinge (150) that will deform plastically but whose strut (155) will remain elastic even though the material is normally considered a plastically deformable material such as stainless steel. A frame (30) constructed with this property will naturally form an oval shape that matches the oval shape of the annulus (10). Alternately, the balloon expandable frame (30) can be designed to retain a round shape by making the strut depth (180) large such that the strut (155) will plastically deform with a high degree of holding force.

The advantages of the present oval bileaflet frame (30) invention over the standard round TAVI devices with round frames are numerous. Standard TAVI devices are known to have perivalvular leaks that occur along the annulus long axis (20) due to the gap that exists between the round frame and the oval annulus (10). Placing a frame (30) with an oval shape into an oval annular space will reduce the amount of perivalvular leaks.

Trileaflet valves do not generally coapt efficiently when the shape of the valve is forced into an oval shape unless the leaflets are of a differing size from one another to accommodate this; the present invention has provided a bileaflet valve (75) that coapts efficiently. The bileaflet valve (75) leaflets (40) of the present invention will provide coaptation of the free edge (65) and a marginal surface (100) or boundary surface of the leaflet with the neighboring leaflet for coaptation even if the shape of the frame (30) is oval and independent of the amount of ovality. The crescent shape of the leaflet of the bileaflet valve (75) of the present invention can coapt very effectively using either a large marginal surface (100) area or a small marginal surface (100) area. The large marginal surface (100) area for valve leaflet coaptation is generated as the frame (30) become more oval in shape such that the frame long axis (35) is significantly larger than the frame small axis.

Trileaflet valves do not coapt efficiently when the diameter of the valve frame is either larger than or smaller that their intended diameter. Therefore when a patient has a larger annulus (10) than anticipated, the physician can either dilate the valve to its intended diameter and risk embolization of the valve or perivalvular leak, or the physician can over-dilate the valve and produce a central reflux of blood through the center of the valve leaflets. If the patient has a smaller annulus (10) than anticipated, the physician can dilate the valve to its intended diameter and risk dissecting the patient's annulus (10) or he can under-dilate the valve frame (30) and obtain poor coaptation of the free edges (65) of the leaflets resulting in excessive wear on the valve leaflets. A bileaflet valve (75) is able to adapt to changes in diameter better than a trileaflet valve. This has been shown by the presence of similar bileaflet valves in the venous system of our body; such veins are able to undergo diameter changes of several times their diameter and still function efficiently. The marginal surface (100) of the bileaflet valve (75) leaflet can automatically adjust to provide efficient leaflet coaptation in a manner that is independent of the frame diameter (120) for diameter changes of 1-5 mm.

Current standard round TAVI devices are required to place adequate force onto the surrounding annulus (10) and native leaflet tissues to ensure that the valve will not embolize. An oval valve that places a more uniform force along the perimeter of the annulus (10) and stenotic leaflets will allow a more gentle force to be applied everywhere along the oval frame perimeter (130). The force of the frame (30) directly onto the membranous septum or the left bundle branch can cause the patient to receive heart block from the implantation of the TAVI device and thereby require the further implantation of a permanent pacemaker. The present oval TAVI device will reduce the outward force requirement and reduce the need for a pacemaker.

The commissures of the present invention are positioned at a location that does not interfere with blood flow through the coronary arteries. The location of the coronary arteries is very rarely found across from each other, and are more typically 120 degrees apart as shown in FIG. 1. The present bileaflet valve (75) commissures are positioned at a location that is not in line with the coronary artery ostii. The commissures of the present bileaflet valve can be placed such that they do not align with the locations of the coronary ostii.

Alternate embodiments of the present invention have been anticipated. Although the primary embodiment of the present invention is an oval-shaped bileaflet aortic valve it is understood that variations of this invention have been anticipated. For example, one could use the oval frame (30) of the present invention with three leaflets (40) and have a trileaflet oval valve. The leaflet structure could be similar to that used in the standard TAVI devices and similar to the trileaflet semilunar valve found in the native aortic valve of the human body. Alternately, one could anticipate a mono leaflet valve having a leaflet shape that is similar to that described in the present invention. The leaflet attached edge (60) could be attached to the frame (30) in a manner similar to that described in the present invention. The free edge (65) of the monoleaflet valve leaflet would coapt or make contact with the opposite wall of the frame (30).

In an alternate embodiment, one could take the bileaflet design of the present invention and apply it to a round frame (30) rather than an oval frame (30). The round frame (30) could be similar to the current cylindrical or round frames currently used in TAVI devices. Any ovalization that occurred during the implantation of the device would be better accommodated by the bileaflet valve (75) leaflets (40). The leaflets (40) would preferably be aligned such that the leaflet long axis (70) aligned with the annulus long axis (20), the leaflet short axis (80) could alternately be aligned with annulus long axis (20) and still provide good leaflet coaptation.

The reference numerals shown in the figures and described in one embodiment of the invention can be applied to alternate embodiments of the invention. It is understood that the present invention is not limited to embodiments presented herein, but includes other embodiments of the invention.

1. An implantable aortic valve device comprising;
A. a frame that is delivered across a stenotic native aortic valve in a small diameter configuration and expandable to a larger diameter configuration to hold the stenotic native aortic valve leaflets to the side, said frame having an oval shape with a long axis and a short axis, said long axis being longer than said short axis,
B. a first and second crescent shaped leaflet, each of said leaflets having a free edge, an attached edge, and a first and second commissure at each longitudinal end of each of said leaflets forming a long axis for each of said leaflets,
C. said leaflets being attached to said frame along said attached edges.

2. The aortic valve device of claim 1 wherein said first and second commissures of each of said leaflets are aligned with a frame axis.

3. The aortic valve device of claim 2 wherein said long axis for said leaflets is aligned with said long axis of said frame.

4. The aortic valve device of claim 2 wherein said long axis for said leaflets is aligned with said short axis of said frame.

5. The aortic valve device of claim 1 wherein said first leaflet has a larger leaflet surface area than said second leaflet.

6. The aortic valve device of claim 1 wherein said frame has an ovality such that the ratio of long axis to short axis ranges from 1.05 to 1.35.

7. The aortic valve device of claim 6 wherein said ratio of long axis to short axis ranges from 1.10-1.20.

8. The aortic valve device of claim 1 wherein said frame has an expansion limiter that prevents expansion of a frame wall structure during expansion deformation of said frame to said larger diameter configuration, thereby forming an oval shape upon expansion.

9. The aortic valve device of claim 1 wherein said frame has a major axis and a minor axis that is substantially equal thereby forming a circular shape for said frame.

10. The aortic valve device of claim 1 further comprising a third crescent shaped leaflet, said third leaflet having a free edge, said free edge of said third leaflet forming a coaptation with said first and said second leaflets.

11. The aortic valve device of claim 1 wherein said frame is formed from a hinge and strut structure such that a hinge width is smaller than said a strut width, a hinge depth is greater than a strut depth, and said hinge length is less than ½ of the strut length, said hinge being plastically deformed during frame expansion, said strut being elastically deformed when it is placed into an oval shape.

12. The aortic valve device of claim 1 wherein said frame is formed from an elastic material, said frame having a hinge and strut structure such that a hinge width is smaller than said a strut width, a hinge depth is greater than a strut depth, and said hinge length is less than ½ of the strut length, said hinge being elastically deformed during frame expansion, said strut being elastically deformed when it is placed into an oval shape, said expansion force of said hinge providing said strut with bending deformation into an oval shape.

13. An implantable aortic valve device intended for transcatheter implant comprising;
A. a frame that is delivered across a stenotic native aortic valve in a small diameter configuration and expandable to a larger diameter configuration to hold the stenotic native aortic valve leaflets to the side, said frame having an oval shape with a long axis and a short axis, said long axis being longer than said short axis,
B. a first and second crescent shaped leaflet, each of said leaflets having a free edge, an attached edge, and a first and second commissure at each longitudinal end of each of said leaflets forming a long axis for each of said leaflets, said long axis for said leaflets being aligned with the long axis of said frame,
C. said leaflets being attached to said frame along said attached edges.

14. An implantable aortic valve device comprising;
A. a frame that is delivered across a stenotic native aortic valve in a small diameter configuration and expandable to a larger diameter configuration to hold the stenotic native aortic valve leaflets to the side, said frame having an oval shape with a long axis and a short axis, said long axis being longer than said short axis,
B. a first and second crescent shaped leaflet, each of said leaflets having a free edge, an attached edge, and a first and second commissure at each longitudinal end of each of said leaflets forming a long axis for each of said leaflets, said long axis for said leaflets being aligned with said short axis of said frame,
C. said leaflets being attached to said frame along said attached edges.
